(19) United States
(12) Patent Application Publication
(10) Pub. No.: US 2016/0000782 A1
(43) Pub. Date: Jan. 7, 2016

(54) PHARMACEUTICAL COMPOSITION CONTAINING ANTIBACTERIAL AGENT

(71) Applicant: KAGOSHIMA UNIVERSITY, Kagoshima-shi, Kagoshima (JP)

(72) Inventors: Hiroshi TAKASHIMA, Kagoshima-shi (JP); Shuji IZUMO, Kagoshima-shi (JP)

(73) Assignee: KAGOSHIMA UNIVERSITY, Kagoshima-shi, Kagoshima (JP)

(21) Appl. No.: 14/769,220

(22) PCT Filed: Feb. 14, 2014
(86) PCT No.: PCT/JP2014/053559

§ 371 Date: Aug. 20, 2015

(30) Foreign Application Priority Data
Feb. 21, 2013 (JP) 2013-032631

(51) Int. Cl. A61K 31/505; A61K 31/42

(52) U.S. Cl. CPC A61K 31/505; A61K 31/42

(57) ABSTRACT

[Problem] The purpose of the present invention is to provide a pharmaceutical composition containing an antibacterial agent, which is effective in elucidating one cause of dementia, and in treatment thereof.

[Solution] The present invention provides a pharmaceutical composition for treatment of dementia, containing an antibacterial agent. The present invention moreover provides a pharmaceutical composition for treatment of extreme halophile infections, containing a folic acid synthesis inhibitor.

### TECHNICAL FIELD

The present invention relates to a pharmaceutical composition comprising an antimicrobial agent.

### BACKGROUND ART

Organisms are classified into three domains: Archaea, Eukaryota, and Bacteria (eubacteria). While one of these domains, Archaea, is known to be relatively similar to Bacteria from a morphological perspective, it is known to be more similar to Eukaryota from a phylogenetic perspective. Archaea is confirmed generally to be present in very unusual environments, such as high-temperature environments such as craters, acidic environments, and salt water. While detection of Archaea in the oral cavity of a patient afflicted with gingivitis had been reported in the past (Methanogenic Archaea and human periodontal disease, Proc. Natl. Acad. Sci., U.S.A., Apr. 20, 2004; 101 (16): 6176 to 81), there had been no reports demonstrating that Archaea would cause a disease in an animal, and Archaea was not considered to be pathogenic to animals, including humans. Accordingly, the correlation between Archaea and animal diseases had not previously been studied to a substantial extent.

Dementia is a concept that has been referred to as “mental deterioration” in the past, and it is a symptom that impairs social life or occupational functioning as a result of cognitive disorder. In addition to lowered memory functions, core symptoms, such as aphasia (difficulty in speech), apraxia (being incapable of motor activity in spite of normal motor functions), agnosia (being incapable of recognition or identification of a substance in spite of normal sensory mechanisms), and deficits in executive function (being incapable of making plans and carrying them out), are observed in patients with dementia. Because of these core symptoms, such as memory disorders, neurological manifestations, such as disorders of emotion and motivation, and behavioral disorders, such as hallucinations, delusions, loitering, and resistance to care, also develop. It is said that the number of patients with dementia in Japan was approximately 2,000,000 as of 2010 and it is presumed that the number of such patients will increase to 3,000,000 or more by 2020.

While cerebral vascular disorders, degenerative diseases such as Alzheimer's disease, normal pressure hydrocephalus, metabolism and nutritional disorders of vitamins and the like, decreased thyroid activity, and other diseases are known as primary causes of dementia, it is often impossible to identify the specific causes thereof. Accordingly, effective therapeutic methods in accordance with such causes are not often established. Therefore, there is a great need for the elucidation of the causes of dementia and the development of therapeutic methods for the same at the social level.

### SUMMARY OF THE INVENTION

### Objects to be Attained by the Invention

The present invention is intended to elucidate a cause of dementia and to provide a pharmaceutical composition that is effective for treatment of dementia.

### Means for Attaining the Objects

The present inventors have conducted concentrated studies in order to identify a cause of dementia. As a result, surprisingly, they discovered that dementia could be caused by infection with Archaea. They also discovered that dementia caused by infection with Archaea could be treated by administration of an antimicrobial agent. This has led to the completion of the present invention.

Specifically, an aspect of the present invention concerns a pharmaceutical composition for treatment of dementia comprising an antimicrobial agent.

According to an embodiment of the present invention, the dementia is associated with encephalitides or encephalomyelitis.

According to an embodiment of the present invention, the dementia is caused by infection with Archaea.

According to an embodiment of the present invention, the antimicrobial agent is an inhibitor of folic acid synthesis.

According to an embodiment of the present invention, the inhibitor of folic acid synthesis is a sulfa drug and/or a 2,4-diaminopyrimidine synthetic antimicrobial agent.

According to an embodiment of the present invention, the sulfa drug is selected from the group consisting of prontosil, sulfamonomethoxine, sulfadiazine, sulfadimethoxine, sulfacetamide, sulfadoxine, sulfanilamide, sulfisomidine, sulfisoxazole, sulfamethoxazole, sulfadimidine, sulfamerazine, sulfaquinoxaline, and pharmacologically acceptable salts thereof, and the 2.4-diaminopyrimidine synthetic antimicrobial agent is selected from the group consisting of brodimoprim, tetroxoprim, trimethoprim, and pharmacologically acceptable salts thereof.

According to an embodiment of the present invention, the Archaea is selected from the group consisting of a methanogen, an extreme halophile, a thermoacidophile, and a hyperthermophile.

According to an embodiment of the present invention, the Archaea is an extreme halophile.

Another aspect of the present invention concerns a pharmaceutical composition for treatment of infection with an extreme halophile comprising an inhibitor of folic acid synthesis.

According to an embodiment of the present invention, the inhibitor of folic acid synthesis is a sulfa drug and/or a 2,4-diaminopyrimidine synthetic antimicrobial agent.

According to an embodiment of the present invention, the sulfa drug is selected from the group consisting of prontosil, sulfamonomethoxine, sulfadiazine, sulfadimethoxine, sulfacetamide, sulfadoxine, sulfanilamide, sulfisomidine, sulfisoxazole, sulfamethoxazole, sulfadimidine, sulfamerazine, sulfaquinoxaline, and pharmacologically acceptable salts thereof, and the 2,4-diaminopyrimidine synthetic antimicrobial agent is selected from the group consisting of brodimoprim, tetroxoprim, trimethoprim, and pharmacologically acceptable salts thereof.

A further aspect of the present invention concerns a method for treatment of dementia comprising a step of administering a therapeutically effective amount of an antimicrobial agent to a subject.

According to an embodiment of the present invention, the dementia is associated with encephalitides or encephalomyelitis.

According to an embodiment of the present invention, the dementia is caused by infection with Archaea.

A further aspect of the present invention concerns a method for treatment of infection with an extreme halophile comprising a step of administering a therapeutically effective amount of an inhibitor of folic acid synthesis to a subject.

### BRIEF DESCRIPTION OF THE DRAWINGS

FIG. 1 shows MRI-FLAIR images of the head of a patient before treatment. FIG. 1A to FIG. 1C show MRI images of Patient 1, FIG. 1D to FIG. 1F and FIG. 1M show MRI images of Patient 2, FIG. 1G to FIG. 11 show MRI images of Patient 3, and FIG. 1J to FIG. 1L show MRI images of Patient 4. In the figure, an arrow indicates the site of a lesion.

FIG. 2 shows changes in MRI images of the head in response to treatment. FIG. 2A to FIG. 2C show MRI images of Patient 1, FIG. 2A shows MRI images after prednisolone administration, FIG. 2B shows MRI images after cerebral biopsy, and FIG. 2C shows MRI images after treatment with an S-T combination agent. FIG. 2D shows MRI images of Patient 2, FIG. 2E shows MRI images of Patient 3, and FIG. 2F shows MRI images of Patient 4. FIG. 2D to FIG. 2F each show an MRI image before treatment on the left and an MRI image after treatment with an S-T combination agent on the right. In the figure, an arrow indicates the site of a lesion, and an arrow head indicates a site of cerebral biopsy.

FIG. 3 shows the results of histological/immunohistochemical observation and electron microscopic observation of tissue sampled via cerebral biopsy. FIG. 3A to FIG. 3H show the results concerning Patient 1. FIG. 3A shows an image obtained by HE staining, FIG. 3B shows an image obtained by PAS staining, FIG. 3C shows an image obtained by grocott staining, FIG. 3D shows an image obtained by immunostaining with the anti-CD4 antibody, FIG. 3E shows an image obtained by immunostaining with the anti-CD8 antibody, FIG. 3F shows an image obtained by immunostaining with the anti-CD68 antibody, and FIG. 3G and FIG. 3H show images obtained by electron microscopy. FIG. 3I shows an image obtained by PAS staining and an image obtained by electron microscopy of the tissue of Patient 2, FIG. 3J shows an image obtained by PAS staining and an image obtained by electron microscopy of the tissue of Patient 3, and FIG. 3K shows an image obtained by PAS staining and an image obtained by electron microscopy of the tissue of Patient 4. In the figure, an arrow head indicates a pathogen structure, and the scale bar is 5 μm.

FIG. 4 shows a summary of the course of treatment for Patient 1. A horizontal axis indicates the number of years since the onset of the disease. An asterisk indicates a time when symptoms of Patient 1 are ameliorated through treatment with an S-T combination agent, and a double asterisk indicates the time when symptoms of Patient 1 are ameliorated again through treatment with an S-T combination agent. In the figure, the term “Dementia/Psychosis” indicates the symptoms of dementia/psychosis, the term “mPSL pulse” indicates the administration of methylprednisolone pulse, “IVIg” indicates high-dose immunoglobulin treatment, “CTRX” indicates administration of ceftriaxone, “TMP-SMX” indicates administration of an S-T combination agent, “PSL” indicates administration of prednisolone, “CSF protein” indicates the quantity of proteins in the spinal fluid, and “CSF cells” indicates the number of cells in the spinal fluid.

FIG. 5 shows cerebral MRI imaging of Patient 4 at the terminal stage. FIG. 5A shows MRI-FLAIR imaging, FIG. 5B shows the results of MRI via gadolinium contrastradiography, and FIG. 5C shows the results of diffusion weighted imaging.

FIG. 6 schematically explicates a method of bioinformatics analysis of the results of sequencing.

FIG. 7 schematically explicates a method of bioinformatics analysis of the results of sequencing.

### EMBODIMENTS FOR CARRYING OUT THE INVENTION

According to the present invention, dementia is a concept that was referred to as “mental deterioration” in the past, and dementia is a disease caused by acquired organic brain dysfunctions. The term “dementia” used in a narrow sense refers to a decline in the mental ability that had once developed normally. The concept of “dementia” in the present invention is not limited thereto, and it includes an extensive range of symptoms, such as cognitive disorders including those of “memory” and “orientation” in addition to “intelligence,” and symptoms associated with cognitive functions of the brain, such as “personality change.” At an early stage, dementia symptoms, such as memory loss, anxiety, lowered motivation, and avoidance of complex tasks, develop, dementia renders patients incapable of taking many actions necessary for daily life as the disease progresses, and symptoms, such as loitering, delusion of injury, and aggressive speech and actions, start to be observed.

In the present invention, methods for identification of dementia are not particularly limited, and known techniques may be employed alone or in combination. For example, cognitive functions can be examined by the mini-mental status examination (MMSE) or with the use of the Revised Hasegawa Dementia Scale (HDS-R). When the Revised Hasegawa Dementia Scale (HDS-R) is employed, for example, cognitive functions are evaluated to have deteriorated when the score is 20 or less, with a full score being 30.

According to the present invention, the term “encephalitides” is a collective designation for inflammatory diseases in the brain, and the term “encephalomyelitis” is a collective designation for inflammatory diseases in the brain and in the spinal cord. In the present invention, the diseases indicated by the term “encephalitides” or “encephalomyelitis” encompass diseases caused by infection with Archaea.

Methods for identification of the development of encephalitides or encephalomyelitis are not particularly limited. For example, the spinal fluid is sampled from a patient, the leukocyte count in the spinal fluid (/mm³) is confirmed to be at a normal level (0 to 5) or higher, and/or the protein level in the spinal fluid (mg/dL) is confirmed to be the normal level (15 to 40) or higher. Thus, the development of encephalitides or encephalomyelitis can be identified. Alternatively, the spinal fluid sampled from a patient may be subjected to, for example, CXCL assays in order to identify the development of encephalitides or encephalomyelitis. CXCL13 is known to become elevated in some types of chronic brain infections, and a higher CXCL13 level indicates development of infectious inflammation in the brain and in the spinal cord. When encephalitides or encephalomyelitis is examined via MRI, for example, MRI can be carried out with the use of a gadolinium contrast agent. Gadolinium is concentrated at a site of continuous inflammation or tumor. If a site enhanced with the use of a gadolinium contrast agent is observed via MRI, accordingly, development of encephalitides or encephalomyelitis can be confirmed.

The term “antimicrobial agent” used in the present invention refers to a chemotherapeutic agent that has activity of suppressing the growth of bacteria (i.e., bacteriostatic activity) or activity of killing bacteria (i.e., bactericidal activity). However, such agent is not necessarily limited to an agent primarily used for bacteriostatic or bactericidal purposes. An agent that is primarily used for other purposes may be used, provided that such agent has bacteriostatic or bactericidal activity. In the present invention, the term “antimicrobial agent” refers to a naturally occurring antimicrobial agent isolated from a microorganism (and it is also referred to as an “antibiotic” ) and also refers to an artificially synthesized antimicrobial agent.

Examples of naturally occurring antimicrobial agents herein include β-lactam, aminoglycoside, tetracycline, lincomycin, chloramphenicol, macrolide, ketolide, polypeptide, and glycopeptide antimicrobial agents. Examples of synthetic antimicrobial agents herein include pyridone carboxylic acid (quinolone), newquinolone, and oxazolidinone antimicrobial agents, sulfa drugs, and trimethoprim.

In the present invention, the term “inhibitor of folic acid synthesis” is a collective designation indicating any drug that inhibits the folic acid synthesis pathway of microorganisms, so as to suppress the growth of microorganisms or kill microorganisms. An inhibitor of folic acid synthesis that is employed in the present invention may inhibit any part of the continuous folic acid synthesis pathway. For example, a sulfa drug that serves as a competitive inhibitor of p-aminobenzoic acid (PABA) to inhibit dihydropteroic acid synthesis and a 2,4-diaminopyrimidine synthetic antimicrobial agent that inhibits dihydrofolate reductase to inhibit tetrahydrofolic acid synthesis can be preferably used.

In the present invention, the term “sulfa drug” is a collective designation indicating any synthetic antimicrobial agent with the sulfonamide (—S(═O)₂—NR₂) site. In the present invention, a sulfa drug is not particularly limited, provided that it serves as a competitive inhibitor of p-aminobenzoic acid (PABA), so as to inhibit dihydropteroic acid synthesis. Preferably, prontosil, sulfamonomethoxine, sulfadiazine, sulfadimethoxine, sulfacetamide, sulfadoxine, sulfanilamide, sulfisomidine, sulfisoxazole, sulfamethoxazole, sulfadimidine, sulfamerazine, sulfaquinoxaline, and pharmacologically acceptable salts thereof can be used. More preferably, sulfamethoxazole and a pharmacologically acceptable salt thereof can be used.

In the present invention, a 2,4-diaminopyrimidine synthetic antimicrobial agent is a pyrimidine-based synthetic antimicrobial agent having amino groups at 2- and 4-positions. In the present invention, a 2,4-diaminopyrimidine synthetic antimicrobial agent is not particularly limited, provided that it inhibits dihydrofolate reductase to inhibit tetrahydrofolic acid synthesis. Preferably, brodimoprim, tetroxoprim, trimethoprim, and pharmacologically acceptable salts thereof can be used. More preferably, trimethoprim and a pharmacologically acceptable salt thereof can be used.

In the present invention, an inhibitor of folic acid synthesis that inhibits a single folic acid synthesis pathway may be used, or several inhibitors that inhibit different regions of the folic acid synthesis pathway may be used in combination. An example of a preferable combination of inhibitors that inhibit different regions of the folic acid synthesis pathway is a combination of a sulfa drug and a 2,4-diaminopyrimidine synthetic antimicrobial agent, with a combination of sulfamethoxazole and trimethoprim being more preferable.

When a combination of a sulfa drug and a 2,4-diaminopyrimidine synthetic antimicrobial agent is used as an inhibitor of folic acid synthesis in the present invention, for example, a combination comprising a sulfa drug and a 2,4-diaminopyrimidine synthetic antimicrobial agent at a ratio of 2:1 to 10:1 can be used. When a combination of sulfamethoxazole and trimethoprim is used, a combination comprising sulfamethoxazole and trimethoprim at a ratio of 2:1 to 10:1 is preferably used, and a combination comprising sulfamethoxazole and trimethoprim at a ratio of 5:1 is more preferably used. In this description, a combination comprising sulfamethoxazole and trimethoprim at a ratio of 5:1 is occasionally referred to as an “S-T combination agent” or “TMP-SMX.” An S-T combination agent exerts synergistic effects through the activity of two types of inhibitors of folic acid synthesis having different action mechanisms.

In the present invention, the term “Archaea” refers to a group of organisms having cell membranes comprising isoprenoid ethers of sn-glycerol-1-phosphate or organisms included therein. Archaea can survive under extreme conditions, such as high salinity, high temperature, high pH, or low pH conditions, they are often separated from, for example, the deep sea, salt water lakes, or hot spring spouts, and they are occasionally separated from animal bodies. While types of Archaea within the scope of the present invention are not particularly limited, examples of such Archaea include a methanogen, an extreme halophile, a thermoacidophile, and a hyperthermophile

In the present invention, the term “methanogen” is a collective designation for Archaea that synthesize methane under anaerobic conditions, and they are occasionally referred to as “methanogenic bacteria” or “methanogenic Archaea.” Methanogens in the present invention are not particularly limited, provided that they have features as described above. Examples thereof include Archaea belonging to Methanobacteria, Methanococci, Methanomicrobia, and Methanopyri of the Euryarchaeota.

While the term “extreme halophiles” is a collective designation for Archaea that require a high sodium chloride concentration for growth, the term “extreme halophiles” used in a broad sense in the present invention occasionally refers to eubacteria that require a high sodium chloride concentration for growth. Extreme halophiles are occasionally referred to as extreme haloarchaea, halophilic bacteria, or haloarchaea. For example, preferable extreme halophiles may grow at a salt concentration of 2.5 M (15 w/v%) or higher, and more preferable extreme halophiles may grow at a salt concentration of 3.3 M (20 w/v%) or higher.

In the present invention, extreme halophiles are not particularly limited, provided that such Archaea have the features described above. An example thereof is Archaea belonging to the Halobacterium of the Euryarchaeota.

In the present invention, the term “thermoacidophiles” is a collective designation for Archaea, for which the optimal growth conditions are acidic or the optimal growth temperature is 45° C. or higher. In the present invention, thermoacidophiles are not particularly limited, provided that such Archaea have the features described above. Examples thereof include Archaea belonging to the Thermoplasmata of the Euryarchaeota and Thermoprotei of the Crenarchaeota.

In the present invention, the term “hyperthermophiles” is a collective designation for Archaea, for which the optimal growth temperature is 80° C. or higher among various types of thermophiles. In the present invention, hyperthermophiles are not particularly limited, provided that such Archaea have the features described above. Examples thereof include Archaea belonging to Thermococci, Archaeoglobi, or Methanopyrus of the Euryarchaeota and Archaea belonging to Crenarchaeota, Aigarchaeota, and Nanoarchaeota.

In the present invention, methods for identification of Archaea as pathogens are not particularly limited, and conventional techniques can be employed alone or in combination. When morphological properties peculiar to Archaea are observed in tissue sampled from a patient, for example, Archaea can be identified histologically. Also, Archaea can be identified by detecting a nucleic acid of Archaea in tissue sampled from a patient, and this technique can be the most conclusive method of identification. When Archaea is to be identified histologically in tissue sampled from a patient, for example, a structure larger than those of common circular or globular bacteria of approximately 1 to 7 μm, which substantially lack an internal structure containing a nucleus and comprise a chromatin-positive substance surrounded by a membrane-like structure, may be identified via toluidine blue/safranin staining or via transmission electron microscopy. Alternatively, Archaea can be histologically distinguished from protozoans or fungi by confirming that such organisms lack complex internal structures such as those observed in eukaryotes while they are positive for PAS or grocott staining.

The most conclusive method for identification of Archaea is DNA sequence identification. When a nucleic acid of Archaea is detected in tissue sampled from a patient, for example, the nucleic acid is extracted from the tissue sampled from the patient in accordance with a conventional technique, PCR is carried out using primers prepared on the basis of the known Archaea genome sequence, and the presence of Archaea can then be determined. In addition, a nucleic acid is extracted from the tissue sampled from the patient, the nucleic acid is subjected to sequencing in accordance with a conventional technique (e.g., the Sanger method) using primers prepared on the basis of the known Archaea genome sequence, and the read sequence is compared with sequences stored in the database. Thus, the presence of Archaea can be determined. Alternatively, a nucleic acid is extracted from tissue sampled from the patient, the nucleotide sequence of the nucleic acid therein is read using a known next-generation sequencing system (e.g., MiSeq® , Illumina), and the read sequence is compared with the nucleotide sequences in the databases that have been decoded in the past, so as to determine that a nucleotide sequence derived from Archaea is present. Thus, the presence of Archaea can be determined. In accordance with a known sequencing method or a known next-generation sequencing system, primers may be prepared from the decoded nucleotide sequence of Archaea, and PCR may be carried out. Thus, the presence of Archaea can be determined with more certainty.

The method of treatment according to the present invention can be applied to any animal targets without particular limitation. If humans are not appropriate, humans can be excluded from the targets. The method of treatment according to the present invention can be applied to targets such as mammalian animals, including mice, rats, dogs, cats, rabbits, cattle, horses, sheep, goats, pigs, monkeys, and humans, and non-mammalian animals including fish, reptiles, amphibians, and birds.

According to the method for treatment of the present invention, methods of administering an antimicrobial agent to a subject are not particularly limited, and a person skilled in the art can select an adequate method of administration in accordance with antimicrobial agent type. In the case of oral administration, for example, intraoral or sublingual administration may be employed. In the case of parenteral administration, for example, intravenous, intramuscular, hypodermic, percutaneous, nasal, or transpulmonary administration may be employed. When an S-T combination agent is to be administered to a subject, for example, oral administration is preferably employed.

According to the method for treatment of the present invention, the dosage of the antimicrobial agent is not particularly limited, and a person skilled in the art can select an adequate amount of the agent in accordance with drug type and patient condition. For example, an S-T combination agent may be administered to a patient in such a manner that active ingredients (that is, 1,600 to 6,400 mg of sulfamethoxazole and 320 to 1,280 mg of trimethoprim) are administered to a patient each day. More preferably, the amounts of active ingredients to be administered to a patient are 3,200 to 4,800 mg of sulfamethoxazole and 640 to 960 mg of trimethoprim per day.

The duration of treatment during which the method for treatment according to the present invention is to be employed is not particularly limited, and such duration can be 6 months or longer, preferably a year or longer, and more preferably as long as two years or longer.

According to the method for treatment of the present invention, the amount of the antimicrobial agent administered to a subject is not necessarily constant during the treatment period. Dosage is preferably reduced by examining improvement in symptoms in accordance with the results of a spinal fluid test, MRI using a gadolinium contrast agent, or the like.

The method for treatment of the present invention is not limited to a method in which an antimicrobial agent is administered to a subject by itself. A person skilled in the art would understand that drugs that are generally administered for the purpose of suppression of inflammation or allergies could be administered to a subject in combination with the antimicrobial agent within the scope of the present invention. Examples of drugs that are generally administered for the purpose of suppression of inflammation or allergies include known steroidal anti-inflammatory agents such as prednisolone, methylprednisolone, dexamethasone, hydrocortisone, hydrocortisone succinate, triamcinolone, triamcinolone acetonide, and betamethasone.

It should be noted that the terms used herein are used to illustrate particular embodiments of the invention and that the scope of the invention is not limited by such terms.

The term “include” used herein is intended to indicate the presence of the matters or items described herein (e.g., members, steps, elements, and numerals), except for cases in which the terms should be understood in a different manner in accordance with context. The use of such term is not intended to exclude the matters or items that are not explicitly described herein (e.g., members, steps, elements, and numerals).

Unless otherwise defined, all terms used herein (including technical and scientific terms) should be understood as a person skilled in the art would generally understand such terms. Unless otherwise defined, the terms used herein should be understood in accordance with the definitions of such terms employed in the relevant technical fields. The terms should not be idealized and should not be understood in an excessively formal manner.

The terms “first,” “second,” and the like are occasionally used in order to express various elements, and the scope of such elements should not be limited by such terms. Since these terms are intended to be used to distinguish one element from another, the first element may be described as the second element and the second element may be described as the first element, for example, within the scope of the present invention.

Hereafter, the present invention is described in greater detail with reference to the examples. It should be noted that various modifications can be made within the scope of the present invention and that the present invention should not be limited to the examples described below.

### EXAMPLES

### (1) Targets of Study

The present inventors conducted studies by targeting 4 subjects with progressive dementia of unknown etiology at the Kagoshima University Medical and Dental Hospital. This study took place after the informed consent of the patients involved had been obtained. Also, this study was carried out with the approval of the ethical review committee of Kagoshima University.

### (2) Patients

### (2-1) Patients' Features

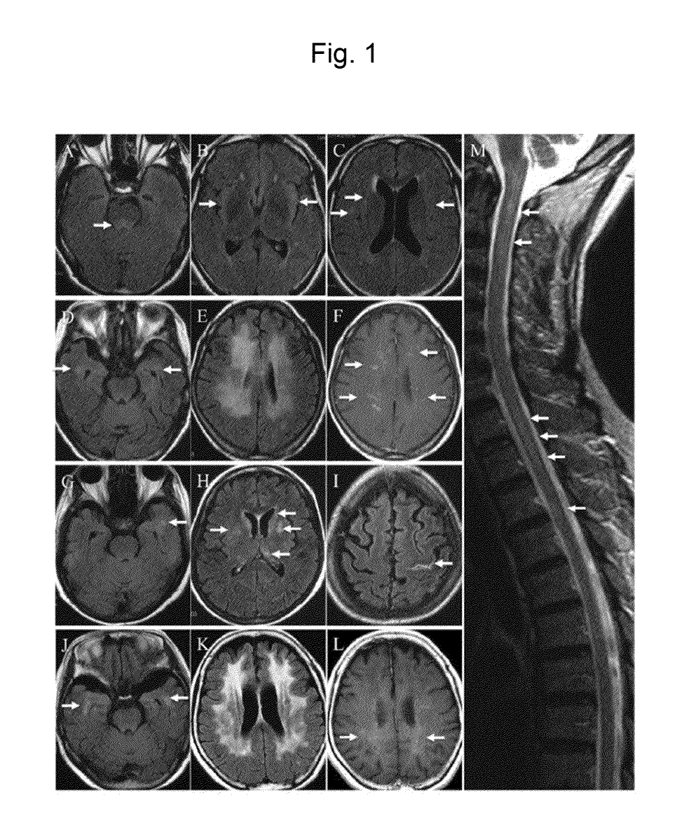

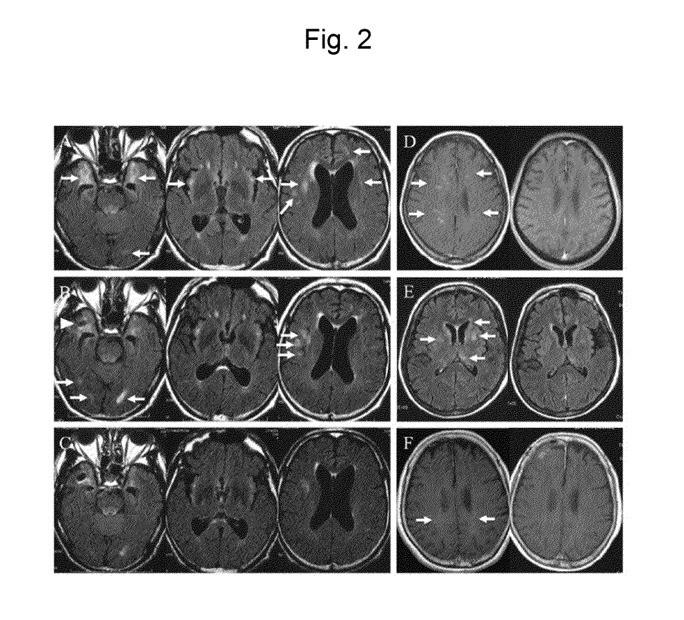

Table 1 shows the features of all patients. All patients developed slowly progressive dementia during the previous year. The age of disease onset was from 47 to 70. All patients had neurological symptoms or involuntary movements of the tongue. Various other symptoms, such as a syndrome of the corticospinal tract, Parkinsonism, and cerebellar ataxia, were observed. Dementia was evaluated using the Revised Hasegawa Dementia Scale (HDS-R), which is the same as that used for the mini-mental status examination (MMSE). The MMSE scores of the subjects were 14 to 18 (maximum score: 30) and the HDS-R scores were 9 to 17 (maximum score: 30). The cerebrospinal fluid test demonstrated an increased cell count and an elevated protein level in the spinal fluid of all patients. The cell count in the cerebrospinal fluid was in the range of 7 to 64 cells/μl, which was increased in all patients (normal level: 0 to 5 cells/μl), and these cells were mainly mononuclear cells. The protein level in CSF was from 45 to 78 mg/dL (normal level: 15 to 40 mg/dL), which was increased in all patients. As a result of MRI of the cerebrospinal cord, lesions with abnormally high signal intensities were observed in the T2-weighted image or the FLAIR image. Lesions were partially enhanced by gadolinium, and activity and inflammation were observed therein. While cerebral disorders were spread over the entire brain, particularly significant lesions were observed in the subcortical white matter of temporal lobe. Disorders were observed in the brain cortex, the basal ganglia, the brain stem, and the spinal cord (FIG. 1). These abnormal MRI results were improved with treatment (FIG. 2).

TABLE 1

| | Patient 1 | Patient 2 | Patient 3 | Patient 4 |
|---|---|---|---|---|
| Age/sex | 47/male | 72/female | 57/female | 70/female |
| Age of disease onset | 47 | 70 | 56 | 68 |
| Chief complaint | Dementia | Dementia/confusion | Dementia | Dementia |
| | Myoclonus | Parkinsonism | Parkinsonism | Loss of frontal lobe |
| | | Disturbance of | Neurological | Cerebellar ataxia |
| | | pyramidal tract | manifestations | |
| Involuntary movements of tongue | Dyskinesis | Unknown | Shivering | Dyskinesis |
| Initial diagnosis | Alzheimer's disease | Intravascular lymphomatosis | Panic disorder | Dementia of unknown etiology |
| Period before initiation of treatment | 11 months | 20 months | 12 months | 27 months |
| with TMP-SMX and effects thereof | Significantly improved | Treatment was uncontinuable | Significantly improved | Treatment was uncontinuable |
| | | due to side effects, in spite | | due to side effects, in spite |
| | | of initial improvement | | of initial improvement |
| Prognosis | Recovery | Slow progression to | Recovery | Death |
| | | equilibrium state | | |
| Immunological test | Negative | Negative | Elevated Interleukin-6 | Elevated Interleukin-6 |
| | | | level in | level in |
| | | | spinal fluid | spinal fluid |
| Revised Hasegawa Dementia Scale | 9 | 13 | 17 | 12 |
| (full score: 30; 20 or lower: | | | | |
| lowered cognition) | | | | |
| MMSE cognition test | No data | 14 | No data | 18 |
| Abnormal MRI results | Abnormal signal | Abnormal signal | Abnormal signal | Abnormal signal |
| | in subcortex | in subcortex | in subcortex | in subcortex |
| | Insular cortex | Deep white matter | Basal ganglia | Deep white matter |
| | Pontine | Spinal cord | Arachnoid | |
| Results of cerebrospinal fluid test | | | | |
| White blood cell count (/mm3) | 27 | 64 | 63 | 7 |
| (normal level: 0 to 5) | | | | |
| Protein (mg/dL) (normal level: 15 | 78 | 66.24 | 74 | 45 |
| to 40) | | | | |
| Sugar (mg/dL) (spinal fluid/serum) | 46/102 | 47/142 | 56/123 | 59/129 |

### (2-2) Patient 1: 47-Year-Old Male

Patient 1 visited, as an outpatient, the Department of Neurology and Geriatrics, Kagoshima University Medical and Dental Hospital on Feb. 9, 2005, and he was hospitalized in the middle of February. A chief complaint was progressive dementia. The patient noticed the increased urination frequency 8 months before he was hospitalized. Patient 1 showed overeating symptoms 5 months thereafter. Conversation and daily activities were decreased as memory disorder and impaired orientation developed. Specialist physicians at two hospitals diagnosed the patient as being afflicted with the Alzheimer's disease. No abnormal results were obtained through magnetic resonance imaging (MRI) or hemanalysis conducted in 3 months after the hospitalization.

After Patient 1 visited the Department of Neurology and Geriatrics, Kagoshima University Medical and Dental Hospital, however, abnormal signals were detected in the dorsal pons, the bilateral insular cortex, and the subcortical lesion through FLAIR-MRI of the head (FIG. 1A to 1C), and an increased cell count was observed in the cerebrospinal fluid (CSF). While the results of physical examination were normal, the results of neurological examination demonstrated that the patient had severely impaired orientation, calculation impairment, and memory disorders. Intermittent myoclonus in the upper extremities and abnormal movements of the tongue were observed. The ability to walk was normal. As a result of physical examination, mild hyperreflexia, a positive sucking reflex sign, a positive Babinski sign, and a positive Chaddock's reflex sign were observed in the extremities. Increased urinary frequency and a feeling of residual urine were also observed.

While the results of the blood test were normal, an increased white blood cell (WBC) count (27 cells/μl and an elevated protein level (78 mg/dL) were observed in the spinal fluid. On the basis of the results demonstrated above, the patient was diagnosed as having infectious diseases or autoimmune diseases.

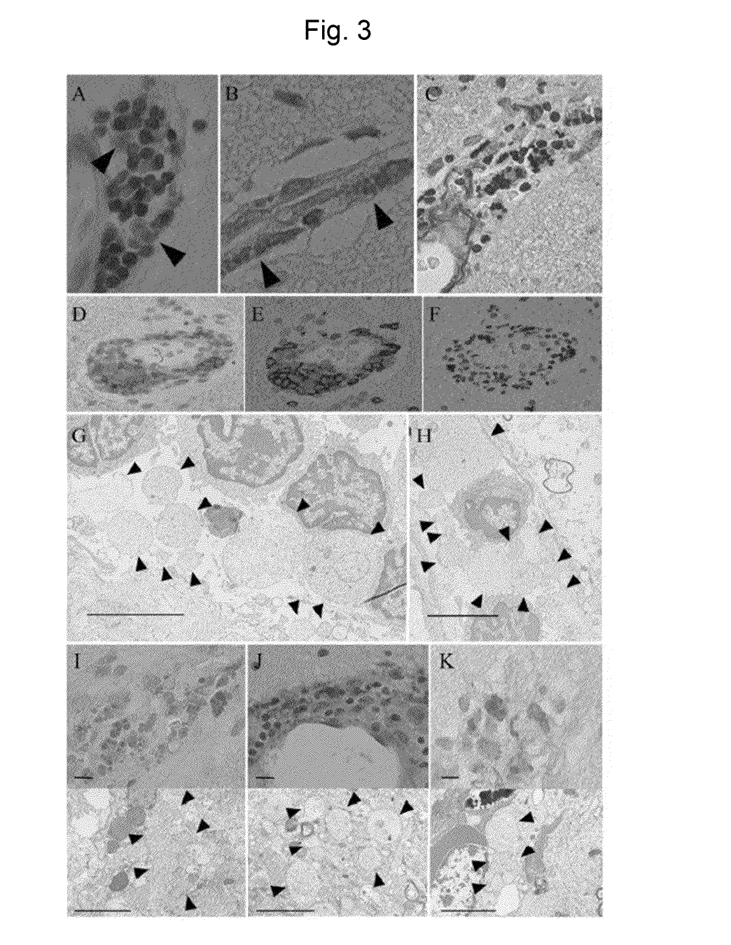

For the initial treatment of infectious diseases or autoimmune encephalitides, ceftriaxone and methylprednisolone were administered to the patient, and symptoms had improved slightly 2 weeks after the initial administration. However, the symptoms tended to progress thereafter, and, in particular, an enlarged lesion was observed in the base of the temporal lobe through brain MRI (FIG. 2A). Since periodic acid-Schiff (PAS)-positive macrophages were observed in the results of cerebral biopsy, the patient was diagnosed as having a particular type of nervous infection (FIG. 3B).

After oval pathogens had been detected via cerebral biopsy, treatment was initiated by administering TMP-SMX (the maximal dose: 12 g/day). TMP-SMX (12 g) contains 4,800 mg of sulfamethoxazole and 960 mg of trimethoprim. TMP-SMX was administered orally to the patient at several separate instances per day. After the initiation of the treatment with TMP-SMX, the symptoms of the patient apparently improved, and the patient had regained writing ability, memory, motivation, and calculation ability 3 months after the initiation of treatment. The patient had become capable of managing daily activities substantially by himself again 5 months after the initiation of treatment with TMP-SMX. He had become capable of performing Internet operations 7 months after the initiation of treatment with TMP-SMX.

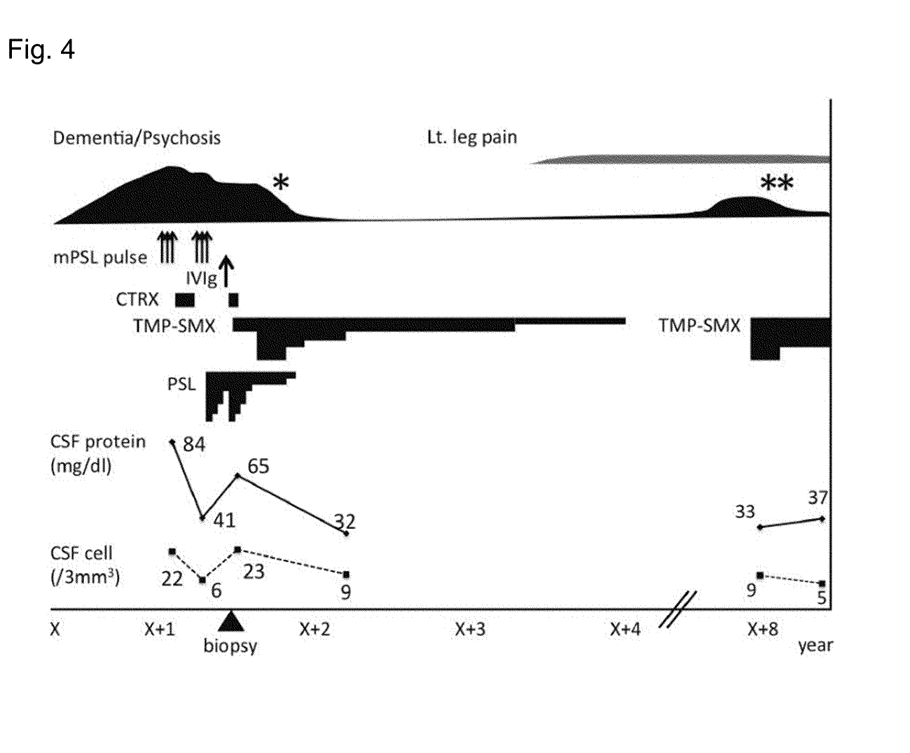

Since memory disorder and impaired orientation recurred 7 years later, TMP-SMX was administered alone (8 g/day), and the patient recovered again. TMP-SMX (8 g) contains 3,200 mg of sulfamethoxazole and 640 mg of trimethoprim. FIG. 4 shows a summary of the clinical course of the patient.

### (2-3) Patient 2: 72-Year-Old Female

Patient 2 is a 72-year-old female with progressive dementia and acroparalysis. Her symptoms were personality change, depression, loss of appetite, and severe malnutrition, and the abilities of walking, cognition, and writing gradually worsened over the following 6 months. In addition, difficulty in conversation and swallowing occurred, and her level of consciousness gradually declined. As a result of neurological examination, symptoms such as semicoma, stiffness in the neck, moderate muscle weakness, shivering, rigidity, hyperreflexia, and the presence of a Babinski sign were observed. Diffuse white matter changes were observed via brain MRI in the cerebral white matter (FIG. 1D to 1F). In the T2 weighed images of the spinal cord in the neck and in the chest obtained by MRI, high-intensity lesions were observed in the center of the spinal cord (FIG. 1M). Discrete punctate lesions were detected in the white matter through MRI using a gadolinium contrast medium. As a result of the spinal fluid examination, it was discovered that white blood cell count had increased.

Since the clinical data for Patient 2 are similar to those for Patient 1, the differential diagnosis of intravascular lymphomatosis or the infectious disease that was the same as that for Patient 1 was made. This diagnosis was supported by the fact that the patient resided in a house approximately 20 km from the coast. As a result of intravenous administration of methylprednisolone (1,000 mg/day), the level of consciousness and the movement disorder improved. While the ability of Patient 2 to hold a conversation was temporarily recovered as a result of treatment with methylprednisolone, the symptoms worsened again. Thereafter, cerebral biopsy was performed, and the pathological findings for Patient 2 were very similar to those for Patient 1 (FIG. 3I). We initiated treatment with TMP-SMX (6 g/day) and dexamethasone, as in the case of Patient 1. She regained consciousness rapidly, and impaired orientation and movement disorder had improved 2 months after the initiation of treatment. However, a liver function failure was caused by TMP-SMX, the treatment was temporarily discontinued, and the symptoms recurred. Thereafter, short-term treatment was repeatedly performed in order to avoid liver function failure caused by TMP-SMX, and the symptoms were then controlled.

### (2-4) Patient 3: 57-Year-Old Female

Patient 3 is a 57-year-old female with progressive dementia and a slight fever. Patient 3 worked in Tokyo, and she visited the house of the parent of Patient 1 twice a year. The house is in a small fishing village on the coast located 10 km south of the house of Patient 1. Patient 1 began to experience general malaise and loss of appetite a year before she visited the hospital. She had symptoms such as impaired orientation, immediate memory loss, and articulation disorder for the following 2 months. She visited the hospital and underwent examinations. An MRI revealed high intensity signals in bilateral pulvinar on T2 weighed images. The symptoms gradually advanced thereafter, and neurological manifestations such as panic reaction, depressive psychosis, and suicidal tendencies were observed. As a result of neurological examination, slow movement and the psychiatric symptoms as described above were observed. As a result of spinal fluid examination, increased lymphocyte count, elevated protein level, and increased IL-6 level were observed (Table 1). For the treatment, methylprednisolone was intravenously administered (1 g/day for 3 consecutive days), the results of the brain MRI worsened, and lesions spread (FIG. 1G to 1I). Symptoms such as a masklike face, muscle rigidity, and involuntary movements of the tongue were observed. These symptoms were similar to those of brain tumors or encephalitides with unknown causes, such as intravascular lymphomatosis or diseases of Patients 1 and 2. Thus, cerebral biopsy was carried out for a definite diagnosis. As a result of the pathological observation of the brain, Patient 3 was considered to have the same diseases as Patients 1 and 2 (FIG. 3J).

While an antibiotic, ceftriaxone, (4 g/day) was administered intravenously for 2 weeks, no improvement was observed. As a result of administration of TMP-SMX and corticosteroids over a period of 3 months, however, pathological and abnormal symptoms of Patient 3 apparently improved. After Patient 3 had received treatment for 3 years, she had sufficiently recovered without recurrence of the disease, in such a manner that she became capable of travelling by herself on a plane. Such conditions have been maintained through continuous administration of TMP-SMX.

### (2-5) : Patient 4

Patient 4 is a 70-year-old female with slight fever and progressive dementia. Patient 4 resided in a town close to the domiciles of Patients 1 to 3. Two years before she was hospitalized, general malaise, loss of appetite, weight loss, and slight fever were observed. She had experienced loss of short-term memory and decreased attention a year before she was hospitalized. As a result of brain MRI, a region exhibiting a high signal intensity was observed in the white matter around the brain ventricle via the FLAIR method (FIG. 1J to 1L). Her ability to walk gradually became unstable 6 months before she was hospitalized. As a result of neurological examination, impaired orientation and constructive apraxia were observed. Also, eye movement disorders, involuntary movements of the tongue, and mild proximal muscle weakness in the lower limbs were observed. As a result of spinal fluid examination, increased mononuclear cell count and elevated protein level were observed (Table 1). While chronic encephalomyelitis and abnormal MRI results of the brain were observed, the causes thereof were unknown. On the basis of the images, the clinical conditions of Patient 4 were considered to be similar to those of Patients 1 to 3. Cerebral biopsy yielded histopathological examination results similar to those of Patients 1 to 3. (FIG. 3K). Thus, oral administration of TMP-SMX (8 g/day) and dexamethasone was initiated.

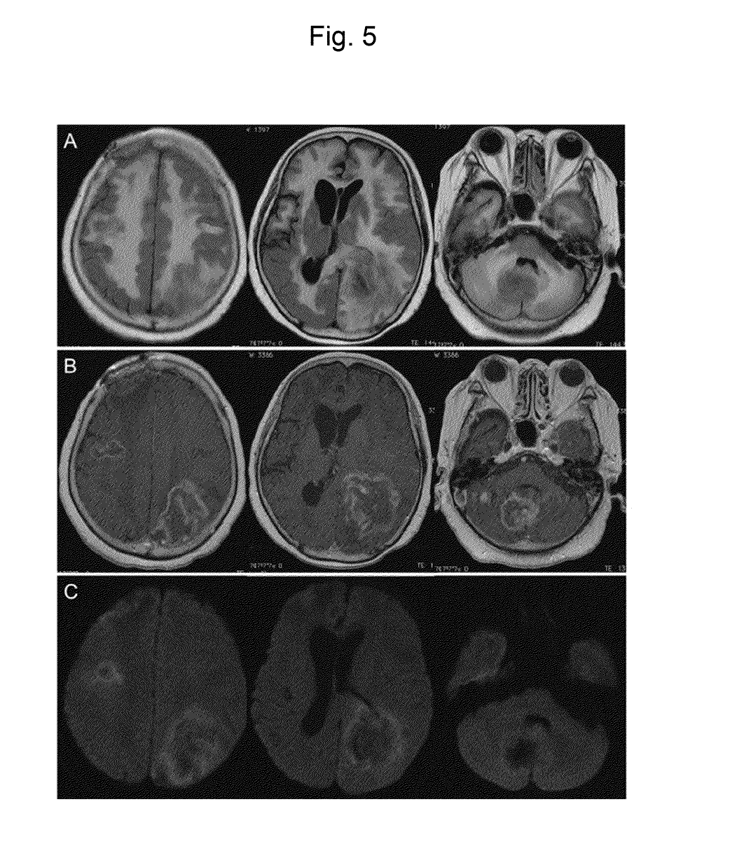

After the treatment, Patient 4 regained sufficient energy to watch TV and to go outside. Involuntary movements of the tongue ceased. In addition, abnormal lesions observed with the use of a contrast medium completely disappeared, and the IL-6 level in the cerebrospinal fluid was reduced to a normal level. As with the case of Patient 2, however, TMP-SMX could not be administered at a high dose to Patient 4 because of a drug-induced liver function failure, and symptoms worsened as a consequence. Patient 4 entered a vegetative state 4 years after the onset of the disease. MRI demonstrated that lesions had spread throughout the brain (FIG. 5), and the patient died.

### (3) Brain Pathological Examination

### (3-1) Method of Brain Pathological Examination

Cerebral biopsy was carried out in order to obtain pathological tissue samples from 4 patients. The tissue blocks sampled via cerebral biopsy were fixed with 4% paraformaldehyde supplemented with 10% neutral-buffered formalin (Wako, Osaka, Japan) and embedded in paraffin. Paraffin sections were then subjected to hematoxylin-eosin (HE) staining, Gram staining, Grocott staining, and PAS staining. The serial tissue sections were subjected to immunohistochemical staining using the anti-CD4 antibody (Nichirei Corporation, Tokyo, Japan) and the anti-CD8 and anti-CD68 antibodies (DAKO, Glostrup, Denmark). The small sample sections were fixed with 3% glutaraldehyde and embedded in Epon 812. The semithin sections for optical microscopy were stained with toluidine blue and safranin. After the exposure of the target tissues to the surface had been confirmed via observation of the semithin sections, ultrathin sections for electron microscopy were then prepared. After the ultrathin sections had been stained with uranyl acetate and lead citrate, observation was carried out under a Hitachi H-7100 electron microscope.

### (3-2) Results of Brain Pathological Examination

In all the tissue samples obtained from the 4 patients via cerebral biopsy, similar histopathological changes, such as an increased number of mononuclear cells (e.g., CD4-positive T lymphocytes (FIG. 3D), CD8-positive T lymphocytes (FIG. 3E), and CD68-positive monocytes (FIG. 3F)) and pathogen invasion in the vicinity of blood vessels (FIG. 3A to FIG. 3C), were observed. (FIG. 3A to FIG. 3H each show histopathological images obtained via cerebral biopsy of Patient 1.)

Subcortical lesions were dominant over cortical lesions. While no macrophage (without foam) aggregates were observed, microabscess formation was observed in Patient 3. Such inflammatory cell invasion was also observed in the subarachnoid cavity. Necrosis, hemorrhage, and demyelination were not apparent. It was difficult to find approximately spherical pathogens (1 to 7 μm) in conventional hematoxylin-eosin-stained sections (FIG. 3A). However, invasion was observed in the cavity in the vicinity of the blood vessels via PAS staining (FIG. 3B), and the cavity was densely stained via Grocott staining (FIG. 3C). The results of Gram staining and immunostaining of the Toxoplasma antigen were negative. Most of these pathogens were localized in the extracellular space. Electron microscopic observation revealed that pathogens would vary in size and that the pathogens were anuclear cells (FIG. 3G and FIG. 3H). While no nuclear membranes or cell walls were observed in these pathogens, a membrane-like intracytoplasmic structure was observed.

In Patient 2 (FIG. 3I), Patient 3 (FIG. 3J), and Patient 4 (FIG. 3K), also, the results of histopathological observation were similar to those observed in Patient 1.

### (4A) Pathogen Nucleic Acid Search Using MiSeq® Next-Generation Sequencing System-1

### (4A-1) DNA or RNA Extraction

DNA was extracted using a DNA extraction kit (Qiagen, Tokyo, Japan) and RNA was extracted using a RecoverAll® total nucleic acid isolation kit (Ambion, Austin, Tex., U.S.A.). DNA and RNA samples were isolated from the brain tissue of the patient via biopsy and a DNA sample was isolated from the spinal fluid. In accordance with the manufacturer's instructions, complementary DNA (cDNA) was amplified using a random primer.

### (4A-2) Sequence Preparation

cDNA was extracted from the brain tissue samples of Patients 3 and 4, whose cerebral biopsy samples were obtained aseptically in accordance with the method described in (4-1). With the use of the Nextera DNA sample preparation kit (Illumina), 50 ng of a cDNA fragment was prepared and the fragment was simultaneously tagged via Nextera transposition. Subsequently, a small DNA fragment, which was shorter than approximately 300 bp, was removed using the AMPure PCR purification system (Agencourt Bioscience, Beverly, Mass., U.S.A.). With the use of the MiSeq® next-generation sequencer (Illumina), the amount of sequence data was doubled via a pair-end sequencing approach to determine a reverse strand.

### (4A-3) Detection of Pathogen Sequence Via Next-Generation Sequencing

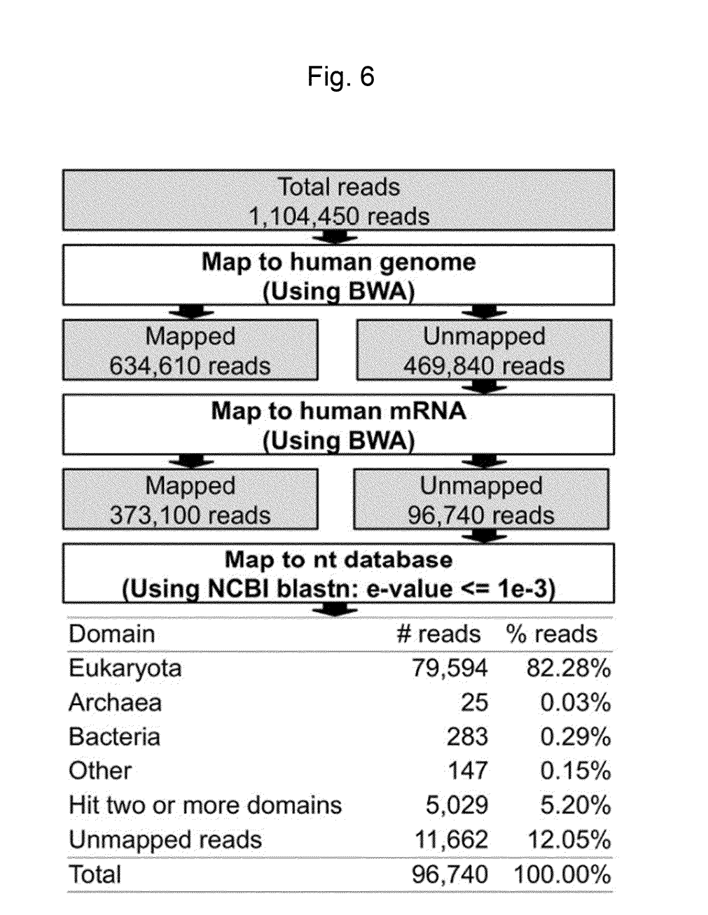

When the average quality value had been reduced to 20 or less in the high-quality region of each read, the 3′-sequence was trimmed. The read sequence was subjected to mapping via two analytical methods. (i) In the case of an rRNA directional search, BLASTN calculation was carried out targeting a cut-off value E of 1e-3 or less and the rRNA sequences stored in the SILVA database (release 111). (ii) When non-human cDNA was to be identified, with the use of a Burrows-Wheeler Aligner (BWA), most human mRNA sequences could be eliminated using a reference sequence used for the human genome (i.e., UCSC:hg19) and human RefSeq (release 54). The read and unmapped sequences were analyzed via BLASTN using a cut-off value E of 1e-3 or less and the NCBI nucleotide sequence database (GenBank release 191). FIG. 6 shows a flow chart showing a summary of the analyses (i) and (ii).

### (4A-A) Sequencing Results

cDNA sequences (1,104,450 reads) were obtained from the brain tissue of Patient 3, and 25 reads thereof were the Archaea-derived sequences (FIG. 6). Organism species having nucleotide sequences exhibiting high homology to the Archaea-derived nucleotide sequences obtained in this experiment were searched for in the database. The results of searches are shown in Table 2 and Table 3. Table 2 shows the results of a BLASTN search in the database and Table 3 shows the results of comparison with the nucleotide sequences of other extreme halophiles stored in the database. The results for one of these reads exhibited a remarkable level of homology to the gene of the GTPase (GenBank Accession Number: YP_{—}002566123.1) observed in an extreme halophile , Halorubrum lacusprofundi (E-value: 2E-19; identity/length:76/85) which is one of Archaea (Table 2 and Table 3). This sequence is not homologous to a bacteria or eukaryote sequence (E-value: 1E-3 or more). The other 24 reads presumed to be derived from Archaea were low-complexity sequences, and, accordingly, these reads could not serve as definitive evidence.

Among the 1,034,377 cDNA sequence reads derived from cerebral biopsy samples of patients with the other disease (i.e., intravascular lymphomatosis) as the negative targets, there was no sequence exhibiting a significant level of homology to the genome or mRNA sequence of Archaea (data not shown).

In addition, cDNA was extracted from the brain tissue of Patient 4 in the same manner and subjected to sequencing in the same manner as in the case of Patient 3. As a result, 2 sequence reads exhibiting high homology to Halobacterium were detected as in the case of Patient 3.

TABLE 2

| | identity/ | | | | | | | | | | |
|---|---|---|---|---|---|---|---|---|---|---|---|
| | aligned | read | | gap | read | read | subject | subject | | | number of |
| hit subject | length | length | mismatch | number | start | end | start | end | e-value | score | best hit |
| gb\|CP001365.1\|Haloruburm | 76/84 | 85 | 8 | 0 | 2 | 85 | 1473401 | 1473484 | 2.00E−19 | 103 | 1 |
| lacusprofundi ATCC 49239 | | | | | | | | | | | |
| chromosome 1, complete | | | | | | | | | | | |
| sequence | | | | | | | | | | | |
| gb\|CP000099.1\|Methanosarcina | 49/50 | 55 | 1 | 0 | 6 | 55 | 2685555 | 2685604 | 4.00E−16 | 91.7 | 1 |
| barkeri str. Fusaro. complete | | | | | | | | | | | |
| genome | | | | | | | | | | | |
| gb\|AE017199.1\|Nanoarchaeum | 33/34 | 52 | 1 | 0 | 16 | 49 | 81234 | 81267 | 1.00E−06 | 60 | 1 |
| equilans Kin4-M, complete | | | | | | | | | | | |
| genome | | | | | | | | | | | |
| gb\|CP002051.1\|Staphylothermus | 32/33 | 54 | 1 | 0 | 16 | 48 | 1338838 | 1338870 | 5.00E−06 | 58 | 1 |
| hellenicus DSM 12710, | | | | | | | | | | | |
| complete genome | | | | | | | | | | | |
| gb\|CP001868.1\|Haloferax | 39/41 | 69 | 1 | 1 | 1 | 40 | 574518 | 574558 | 7.00E−06 | 58 | 1 |
| mediterranei ATCC 33500, | | | | | | | | | | | |
| complete genome | | | | | | | | | | | |
| emb\|FP565147.1\|uncultured | 40/44 | 48 | 4 | 0 | 5 | 48 | 2135176 | 2135133 | 2.00E−05 | 56 | 1 |
| arhaeon ANME-1, | | | | | | | | | | | |
| unordered contigs | | | | | | | | | | | |
| gb\|CP000505.1\|Thermofilum | 31/32 | 62 | 1 | 0 | 23 | 54 | 962266 | 962297 | 2.00E−05 | 56 | 1 |
| pendens Hrk 5, | | | | | | | | | | | |
| complete genome | | | | | | | | | | | |
| gb\|CP002057.1\|Methanococcus | 29/29 | 132 | 0 | 0 | 68 | 96 | 1733854 | 1733826 | 2.00E−05 | 58 | 1 |
| voltae A3, complete genome | | | | | | | | | | | |
| gb\|CP000505.1\|Thermofilum | 31/32 | 47 | 1 | 0 | 13 | 44 | 962266 | 962297 | 2.00E−05 | 56 | 1 |
| pendens Hrk 5, | | | | | | | | | | | |
| complete genome | | | | | | | | | | | |
| gb\|CP000505.1\|Thermofilum | 31/32 | 74 | 1 | 0 | 16 | 47 | 962266 | 962297 | 3.00E−05 | 56 | 1 |
| pendens Hrk 5, | | | | | | | | | | | |
| complete genome | | | | | | | | | | | |
| gb\|CP000505.1\|Thermofilum | 31/32 | 66 | 1 | 0 | 9 | 40 | 962266 | 962297 | 3.00E−05 | 56 | 1 |
| pendens Hrk 5, | | | | | | | | | | | |
| complete genome | | | | | | | | | | | |
| gb\|CP000505.1\|Thermofilum | 31/32 | 65 | 1 | 0 | 6 | 37 | 962266 | 962297 | 3.00E−05 | 56 | 1 |
| pendens Hrk 5, | | | | | | | | | | | |
| complete genome | | | | | | | | | | | |
| gb\|CP000505.1\|Thermofilum | 31/32 | 74 | 1 | 0 | 29 | 60 | 962266 | 962297 | 3.00E−05 | 56 | 1 |
| pendens Hrk 5, | | | | | | | | | | | |
| complete genome | | | | | | | | | | | |
| gb\|AE010299.1\|Methanosarcina | 30/31 | 38 | 1 | 0 | 1 | 31 | 5248539 | 5248509 | 4.00E−05 | 54 | 1 |
| acetivorans str. C2A, complete genome | | | | | | | | | | | |
| gb\|CP000505.1\|Thermofilum | 31/32 | 91 | 1 | 0 | 32 | 63 | 962266 | 962297 | 4.00E−05 | 56 | 1 |
| pendens Hrk 5, | | | | | | | | | | | |
| complete genome | | | | | | | | | | | |
| gb\|CP000505.1\|Thermofilum | 31/32 | 93 | 1 | 0 | 34 | 65 | 962266 | 962297 | 4.00E−05 | 56 | 1 |
| pendens Hrk 5, | | | | | | | | | | | |
| complete genome | | | | | | | | | | | |
| gb\|CP000505.1\|Thermofilum | 31/32 | 113 | 1 | 0 | 36 | 67 | 962266 | 962297 | 5.00E−05 | 56 | 1 |
| pendens Hrk 5, | | | | | | | | | | | |
| complete genome | | | | | | | | | | | |
| gb\|CP000505.1\|Thermofilum | 31/32 | 133 | 1 | 0 | 42 | 73 | 962266 | 962297 | 6.00E−05 | 56 | 1 |
| pendens Hrk 5, | | | | | | | | | | | |
| complete genome | | | | | | | | | | | |
| gb\|CP000505.1\|Thermofilum | 31/32 | 126 | 1 | 0 | 27 | 58 | 962266 | 962297 | 6.00E−05 | 56 | 1 |
| pendens Hrk 5, | | | | | | | | | | | |
| complete genome | | | | | | | | | | | |
| dbj\|BA000023.2\|Sulfolobus | 27/27 | 53 | 0 | 0 | 22 | 48 | 1889036 | 1889010 | 7.00E−05 | 54 | 1 |
| lokodaii str. 7 DNA, complete genome | | | | | | | | | | | |
| gb\|CP002952.1\|Thermococcus sp. | 33/35 | 64 | 2 | 0 | 5 | 39 | 1759998 | 1760032 | 1.00E−04 | 54 | 1 |
| AM4, complete genome | | | | | | | | | | | |
| gb\|CP000561.1\|Pyrobaculum | 30/31 | 119 | 1 | 0 | 82 | 112 | 566539 | 566589 | 2.00E−04 | 54 | 1 |
| calidifontis JCM 11548, | | | | | | | | | | | |
| complete genome | | | | | | | | | | | |
| gb\|CP003534.1\|Pyrococcus sp. ST04, | 27/27 | 89 | 0 | 0 | 12 | 38 | 642602 | 642628 | 2.00E−04 | 54 | 1 |
| complete genome | | | | | | | | | | | |
| gb\|CP002062.1\|Halalkalicoccus | 26/26 | 80 | 0 | 0 | 55 | 80 | 2056537 | 2056562 | 5.00E−04 | 52 | 1 |
| jeotgali B3, | | | | | | | | | | | |
| complete genome | | | | | | | | | | | |
| gb\|AE010299.1\|Methanosarcina | 29/30 | 109 | 1 | 0 | 17 | 46 | 1927925 | 1927896 | 8.00E−04 | 52 | 2 |
| acctivorans str. C2A, complete genome | | | | | | | | | | | |
| gb\|AE319044.1\|Methanosarcina | 29/30 | 109 | 1 | 0 | 17 | 46 | 680 | 709 | 8.00E−04 | 52 | 2 |
| acctivorans strain C2A | | | | | | | | | | | |
| MtaF (matF) and MtaG (mtaG) | | | | | | | | | | | |
| genes, complete cds | | | | | | | | | | | |

TABLE 3

| | | | | * | | | * | | | | | | | | | * | | | * | | |
|---|---|---|---|---|---|---|---|---|---|---|---|---|---|---|---|---|---|---|---|---|---|
| cDNA extracted from affected brain | C | C | C | G | C | G | G | C | C | C | T | C | G | T | G | G | G | C | G | C | C |
| Halorabrum lacusprojundi (e-value = 4E−19) | — | | | | | | C | | | | | | | | | | | | | | |
| Halotkalic occus fcotgali | | | | T | | | | | | | | | | | | | | | | | |
| Halomacrobrum mukohataei | — | | | C | | | A | | | | | | | | | | | | A | | |
| Haloarcula hispanlca | — | | | | | | | | | | | | | | | T | | | | | |
| Halobacterum salomaram | | | | | | | — | | | | | | | | | | | | | | |
| Halopiger xanaduensis | — | — | — | — | — | — | | | | | | | | | | | | | | | |
| Haloterrigena turkmenica | | | | | | | — | | | | | | | | | | | | | | |
| Haloktrcula auarsmotui | | | | | | | C | | | | | | | | | T | | | | | |
| | | * | * | | * | * | * | * | * | * | * | | | | | | | | * | | * |
| cDNA extracted from affected brain | A | C | G | A | G | C | T | G | G | A | C | C | G | G | G | A | C | G | A | A | C |
| Halorabrum lacusproftokli re-value = 4E−19) | | | C | | T | | | T | C | | | | | | | | | | | | G |
| Halalkolicoccus leatgali | | T | C | | | | | C | | | | | | | | | | | T | | G |
| Halomicrobium mukohataei | | | | | | A | C | | C | | | | | | | | | | | | |
| Haloarcnia hispanica | | T | | | | | | C | C | | | | | | | | | | T | | G |
| Halobacterium salinarum | | | | | | | | T | C | G | T | | | | | | | | | | |
| Halopiger xanabensis | | | | | | T | | T | C | G | T | | | | | | | | | | |
| Haloterrigena tarkmenica | | | | | | T | | | | | | | | | | | | | | | |
| Haloarcula marismarni | | | | | | | | T | C | G | T | | | | | | | | | | |
| | * | | | * | | * | | | | * | | | * | | | * | | | | | |
| cDNA extracted from affected brain | C | G | G | G | A | C | G | A | G | C | C | C | G | G | C | G | A | C | G | T | C |
| Halorabrum lacusprojundi (e-value = 4E−19) | | | | | | | | | | G | | | | | | | | | | | |
| Halotkalic occus fcotgali | | | | | | | | | | G | | | | | | C | | | | | |
| Halomacrobrum mukohataei | G | | | | | | | | | G | | | | | | C | | | | | |
| Haloarcula hispanlca | G | | | | | | | | | G | | | | | | C | | | | | |
| Halobacterum salomaram | | | | | | T | | | | G | | | C | | | | | | | | |
| Halopiger xaroduensis | G | | | A | | T | | | | G | | | | | | | | | | | |
| Haloterrigena turkmenica | G | | | A | | T | | | | A | | | | | | | | | | | |
| Haloktrcula auarsmotui | | | | | | | | | | | | | | | | | | | | | |
| | | | * | * | | * | | | * | | | * | * | * | | | | | | | |
| cDNA extracted from affected brain | C | G | G | G | T | G | C | C | C | T | C | G | C | G | G | C | A | G | A | C | G |
| Halorabrum lacusproftokli re-value = 4E−19) | | | | | | | | | A | | | | | | | | | | | | |
| Halalkolicoccus leatgali | | | T | T | | T | | | | | | | T | | | | | | — | — | — |
| Halomicrobium mukohataei | | | | | | | | | | | | — | — | — | — | — | — | — | — | — | — |
| Haloarcnia hispanica | | | C | T | | | | | G | | | C | | | | | | | — | — | — |
| Halobacterium salinarum | | | C | | | | | | G | | | C | T | C | | | | | | — | — |
| Halopiger xanaduensis | | | | | | | | | — | — | — | — | — | — | — | — | — | — | — | — | — |
| Haloterrigena tarkmenica | | | | | | | | | — | — | — | — | — | — | — | — | — | — | — | — | — |
| Haloarcula marismarni | | | | | | C | | | — | — | — | — | — | — | — | — | — | — | — | — | — |

### (4B) Pathogen Nucleic Acid Search Using the MiSeq® Next-Generation Sequencing System-2

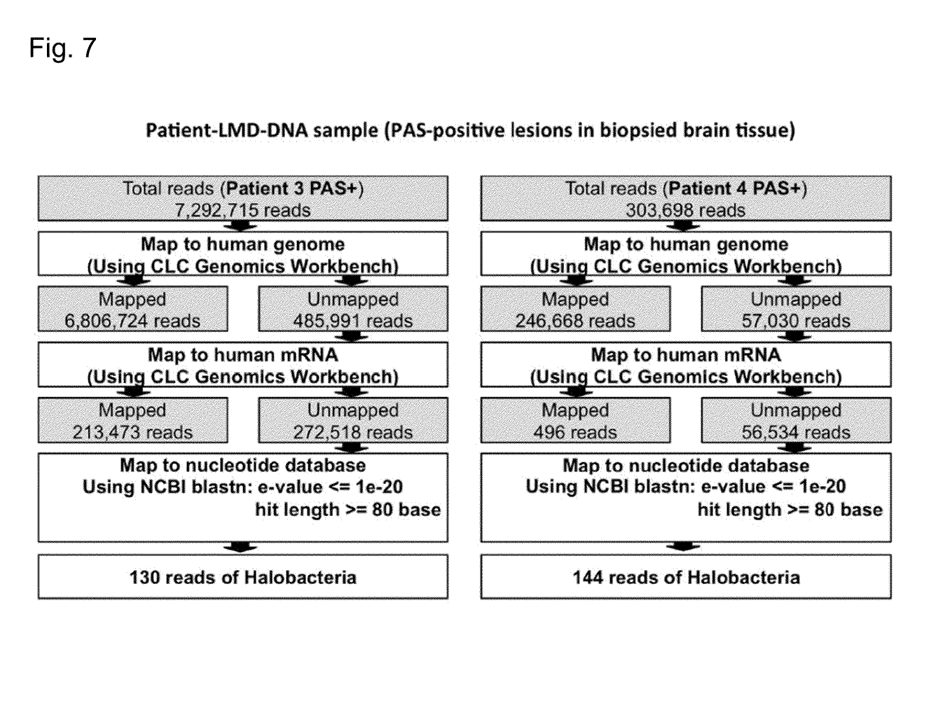

With the use of the MiSeq® next-generation sequencing system, 7,292,715 DNA sequence reads were obtained from 36 spots collected from the samples of Patient 3 via microdissection. Sequences unmapped to the human genome and human mRNA were selected from these DNA sequences using the CLC Genomics Workbench. The selected DNA sequences were subjected to search under conditions of an E-value of 1E-20 or less, a hit length of more than 80, and homology of 70% or higher. As a result, 130 reads were found to exhibit very high homology to the Halobacterium sequence (FIG. 7). Such sequences were not homologous to the sequences of bacteria or eukaryotes (E-value of 1E-3 or more).

In addition, 303,698 DNA sequence reads were obtained from 36 spots collected from the samples of Patient 4 via microdissection using the MiSeq® next-generation sequencing system. Sequences unmapped to the human genome and human mRNA were selected from these DNA sequences using the CLC Genomics Workbench. The selected DNA sequences were subjected to search under the same conditions as those employed for Patient 3. As a result, 144 sequence reads were found to exhibit very high homology to the Halobacterium sequence, including Halorubrum lacusprofundi and Halophilic archaeon (FIG. 7).

Among the 4,760,858 cDNA sequence reads, the 5,259,934 cDNA sequence reads, and the 5,027,830 cDNA sequence reads obtained from cerebral biopsy samples collected from patients with other diseases (i.e., papillary meningioma, intravascular lymphomatosis, and glioblastoma), no sequence exhibiting significant homology to the genome or mRNA of Archaea was detected (data not shown).

### (5) CXCL13 Assay

### (5-1) Method of CXCL13 Assay

CXCL13 is a human B lymphocyte chemoattractant, and it is a very effective attractant for human B lymphocytes in the blood. CXCL13 was assayed using the ELISA kit (R&D Systems, Abingdon, United Kingdom) in accordance with the method recommended by the manufacturer.

### (5-2) Results of CXCL Assay

The levels of chemokine (CXCL13) in the spinal fluid samples of the 4 patients were assayed. Before the treatment was initiated, the CXCL13 levels had become elevated to 500 pg/ml or higher in all the patients. After the treatment with TMP-SMX in combination with corticosteroid, the CXCL13 levels had fallen to 466 pg/ml (Patient 1), 211 pg/ml (Patient 2), 30.7 pg/ml (Patient 3), and 22.2 pg/ml (Patient 4), respectively.

### (6) Summary of Results

The pathogens causing dementia have circular or spherical shapes with diameters of 1 to 7μm, they have substantially no internal structures, no nuclear structures are observed, and chromatin-positive substances exist in cells surrounded by a membrane-like structure. On the basis of such findings, the pathogens of interest can be distinguished from bacteria and eukaryotes. While the pathogens causing dementia are positive for PAS staining and Grocott staining, they are free of internal structures or cell walls, unlike protozoa. Thus, such pathogens can also be distinguished from fungi or protozoa. In addition, such pathogens do not cause nerve cell swelling, an image indicating nuclear destruction, and intracellular inclusion. Thus, such pathogens can be distinguished from virus infections. Further, the disease responded to the treatment with an antimicrobial agent; i.e., an S-T combination agent. Since there are no organisms except for Archaea that exhibit such morphological and pathological properties, the pathogens causing the disease were identified as Archaea.

In addition, RNA was extracted from the brain tissue samples aseptically extracted from two patients (Patient 3 and Patient 4), and cDNA thereof was subjected to sequence analysis using a next-generation sequencer. As a result, gene sequences derived from extreme halophiles (Halobacteriumaea) were detected in both patients. This demonstrates that the pathogens causing the dementia were Archaea. As a result of the analysis, the DNA sequences of Archaea obtained from the brain samples of the two patients were found to be novel sequences while they were homologous to bacteria that had previously been reported. Thus, the pathogens causing the dementia were found to be extreme halophiles or Archaea very similar to the extreme halophiles.

### (7) Conclusions

As a result of the study described above, surprisingly, the present inventors discovered that a cause of dementia could be infection with Archaea. They also discovered that the dementia caused by infection with Archaea could be treated with the administration of an antimicrobial agent to a subject.

In addition, the present inventors discovered as a result of this study that Archaea, which had not previously been considered to be pathogenic to mammalians, can cause diseases in animals. They further discovered that diseases caused by Archaea can be treated with the administration of an antimicrobial agent to a subject.

### INDUSTRIAL APPLICABILITY

The present invention enabled the treatment of a certain type of dementia that had been previously incurable.

According to the present invention, also, the new concept of “diseases caused by infection with Archaea,” which had not been known in the past, was elucidated, and such discovery enabled the treatment of such diseases through the administration of an antimicrobial agent.

1-11. (canceled)

12. A method for treatment of dementia caused by infection with Archaea comprising a step of administering a therapeutically effective amount of an antimicrobial agent to a subject.

13. The method for treatment according to claim 12, wherein the dementia is associated with encephalitides or encephalomyelitis.

14. (canceled)

15. A method for treatment of infection with an extreme halophile comprising a step of administering a therapeutically effective amount of an inhibitor of folic acid synthesis to a subject.

16. The method according to claim 12, wherein the antimicrobial agent is an inhibitor of folic acid synthesis.

17. The method according to claim 16, wherein the inhibitor of folic acid synthesis is a sulfa drug and/or a 2,4-diaminopyrimidine synthetic antimicrobial agent.

18. The method according to claim 17, wherein the sulfa drug is selected from the group consisting of prontosil, sulfamonomethoxine, sulfadiazine, sulfadimethoxine, sulfacetamide, sulfadoxine, sulfanilamide, sulfisomidine, sulfisoxazole, sulfamethoxazole, sulfadimidine, sulfamerazine, sulfaquinoxaline, and pharmacologically acceptable salts thereof and the 2,4-diaminopyrimidine synthetic antimicrobial agent is selected from the group consisting of brodimoprim, tetroxoprim, trimethoprim, and pharmacologically acceptable salts thereof.

19. The method according to claim 12, wherein the Archaea is selected from the group consisting of a methanogen, an extreme halophile, a thermoacidophile, and a hyperthermophile.

20. The method according to claim 19, wherein the Archaea is an extreme halophile.

21. The method according to claim 15, wherein the inhibitor of folic acid synthesis is a sulfa drug and/or a 2,4-diaminopyrimidine synthetic antimicrobial agent.

22. The method according to claim 21, wherein the sulfa drug is selected from the group consisting of prontosil, sulfamonomethoxine, sulfadiazine, sulfadimethoxine, sulfacetamide, sulfadoxine, sulfanilamide, sulfisomidine, sulfisoxazole, sulfamethoxazole, sulfadimidine, sulfamerazine, sulfaquinoxaline, and pharmacologically acceptable salts thereof and the 2,4-diaminopyrimidine synthetic antimicrobial agent is selected from the group consisting of brodimoprim, tetroxoprim, trimethoprim, and pharmacologically acceptable salts thereof.
